(19) United States
(12) Patent Application Publication
(10) Pub. No.: US 2012/0010708 A1
(43) Pub. Date: Jan. 12, 2012

(54) AMNION AND CHORION REPLACEMENT COVER AND USES THEREOF IN SURGICAL REPAIR OF MUSCLES

(75) Inventors: Robin R. YOUNG, Wayne, PA (US); Richard M. JAY, Philadelphia, PA (US)

(73) Assignee: AFCELL MEDICAL, Parsippany, NJ (US)

(21) Appl. No.: 13/178,980

(22) Filed: Jul. 8, 2011

Related U.S. Application Data
(60) Provisional application No. 61/362,447, filed on Jul. 8, 2010.

(51) Int. Cl. A61F 2/08

(52) U.S. Cl. 623/14.13

(57) ABSTRACT

Improved methods for a surgical repair of a muscle are described. The improvement includes covering a damaged site of muscle with at least one of an amniotic fluid and a replacement cover for muscle sheath prior to wound closing during the surgery. The replacement cover contains at least one layer of human amnion and chorion tissues and is adapted to a shape appropriate for enclosing the muscle. The methods reduce inflammation, inhibit fibrosis, scarring, fibroblast proliferation and post-operative infection, while also promote more rapid healing and smooth gliding of the affected muscle against adjacent structures. Related replacement covers, kits and methods of preparation are also described.

### CROSS-REFERENCE TO RELATED APPLICATIONS

This application is entitled to priority pursuant to 35 U.S.C. §119(e) to U.S. Provisional Patent Application No. 61/362,447, filed Jul. 8, 2010 which is hereby incorporated by reference herein in its entirety.

### BACKGROUND OF THE INVENTION

1. Field of Invention

This invention relates to methods and products for improving the surgical repair of muscles, such as torn, ruptured, injured, deformed or pathological diseased muscles. The methods involve the application of a replacement cover of muscle sheath comprising at least one of processed human amnion and chorion tissues to reduce inflammation, inhibit fibrosis, scarring, fibroblast proliferation and post-operative infection while also promoting more rapid healing and smooth gliding of the affected muscle against adjacent structures.

2. Background of the Invention

Skeletal muscle is comprised of contractile filaments that move past each other changing in length to produce force and cause motion throughout the body. There exists a thin fascia membrane of collagen and fibronenctin tissues, called epimysium, which coats and protects muscle fibers. Specifically, this thin membrane protects the muscle from tethering to surrounding bone and tissue and allows it to move and glide freely against adjacent structures. The epimysium also protects the muscle fibers from microbial invasion and inflammation.

Skeletal muscle is comprised of several structures which are responsible for body movement. Among those are the myofibrils which consist of protein filaments and are comprised of thin filaments, actin, and a thick filament called myosin. Bundles of myofibrils are anchored to the inside of a cell membrane called the sarcolemma and at each end, the outer surface of the sarcolemma fuses to the collagen fibers of the muscles. The endomysium, a layer of connective tissue which contains capillaries, nerves and lymphatics, ensheaths the sarcolemma. The perimysium bundles these muscle fibers into fascicles. The fascicles are enclosed in a sheath of epimysium to form a muscle. The epimysium protects the muscle from the friction caused by surrounding tissue and bone.

Patients whose muscle(s) have been torn lose the protective function of the epimysium which, in turn, reduces the ability of the patient's muscle to move and glide freely between articulating and adjacent tissues. Specifically, when the muscle sheath is injured, stressed or traumatized, it typically reacts by increasing the tensional forces making a “sling” over the injured muscle. The muscle sheath can also respond to trauma by “gluing” affected areas of the site of muscle injury. After the muscle trauma, the fascia sheathing sometimes “forgets” to unglue and patients then experience adhesion and scarring at the muscle injury site. In those cases, the muscle no longer slides and adjacent structures painfully tether and tug at each other.

The most common torn muscle is the hamstring muscle complex (HMC). Most common in athletes, this injury causes pain, lose of strength and time lost from activity. As part of the injury, the epimysium is torn. Post operatively, one of the main purposes of exercise rehabilitation is to move the muscle, even as it is healing, in order to reduce the incidence of internal scarring and tethering of the muscle. Failure to properly exercise the healing muscle will result in reduced range of motion in the patient. Even with proper rehabilitation, however, most patients still do not achieve full range of motion following muscle tears because of the damage to the muscle sheathing. Recurrent tears may occur near the tethered fibers since more force is required to contract the muscle.

Methods and products which would effectively inhibit fibroblast formation, scarring and adhesion formation would be useful for treating torn or otherwise injured muscles. Currently there is no product available for use during surgical repair of torn muscles tissue which would restore the torn muscle sheathing and improve post operative outcomes.

The amnion is a thin, cellular, extra-embryonic membrane that forms the inner membrane of a closed sac surrounding and protecting an embryo in reptiles, birds, and mammals. The sac contains the fetus and amniotic fluid or liquor amnii, in which the embryo is immersed, nourished and protected. Typically, the amnion is a tough, transparent, nerve-free, and nonvascular membrane consisting of two layers of cells: an inner, single-cell-thick layer of ectodermal epithelium and an outer covering of mesodermal, connective, and specialized smooth muscular tissue. In the later stages of pregnancy, the amnion expands to come in contact with the inner wall of the chorion creating the appearance of a thin wall of the sac extending from the margin of the placenta. The amnion and chorion are closely applied, though not fused, to one another and to the wall of the uterus. Thus, at the later stage of gestation, the fetal membranes are composed of two principal layers: the outer chorion that is in contact with maternal cells and the inner amnion that is bathed by amniotic fluid. The amnion has multiple functions, i.e., as a covering epithelium, as an active secretary epithelium, and for intense intercellular and transcellular transport. Before or during labor, the sac breaks and the fluid drains out. Typically, the remnants of the sac membranes are observed as the white fringe lining the inner cavity of the placenta expelled after birth. The amnion can be stripped off from the placenta. The amnion has a basement membrane side and a stroma side. The fetal membrane including amnion and chorion has been used in surgeries documented as early as 1910. See Trelford et al., 1979, Am J Obstet Gynecol, 134:833-845. Amnioplastin, an isolated and chemically processed amniotic membrane, was used for continual dural repair, peripheral nerve injuries, conjunctival graft and flexor and tendon repair. See e.g., Chao et al., 1940, The British Medical Journal, March 30. The amnion has been used for multiple medical purposes, e.g., as a graft in surgical reconstruction forming artificial vaginas or over the surgical defect of total glossectomy, as a dressing for burns, on full-thickness skin wounds or in omphalocele, and in the prevention of meningocerebral adhesions following head injury or tissue adhesion in abdominal and pelvic surgery.

In 1962, the fetal membrane was used to treat pelvic basins after total exenteration in dogs, however, trials in human proved disappointing.

In recent years, there have been renewed interests in the application of amnion in ocular surface reconstruction, for example, as an allograph for repairing corneal defects. See, for example, Tsai and Tseng, Cornea. 1994 September; 13(5):389-400; and Dua et al., Br. J Ophthalmol 1999, 83:748-20 752. In addition, amnion and amniotic fluid have recently been used as sources of placental stem cells. See, e.g., U.S. Pat. No. 7,255,879 and WO 200073421.

The role of the amniotic membrane was investigated in chickens with regard to the prevention of adhesion formation following tendon repair in zone II. Results of histologic examination demonstrated that use of the amniotic membrane significantly reduced the amount of adhesion compared with the other groups. Three months after implantation no remnants of amniotic membrane could be identified at the tendon repair site. Demirkan et al., Archives of Orthopaedic and Trauma Surgery, 2002, vol. 122: 396-399.

Despite the clinical and published record regarding the safety and efficacy of amnion in broad surgical use, issues regarding reproducibility, safety and the precise form of amnion for each prospective indication have prevented amnion from achieving broad commercial distribution.

There is a need of improved methods and products for treating torn and otherwise injured muscles that would effectively reduce inflammation, inhibit fibroblast formation, scarring, adhesion formation and post-operative infection, while also promote more rapid healing and smooth gliding of the affected muscle against adjacent structures. The present invention relates to such improved methods and products.

### BRIEF SUMMARY OF THE INVENTION

In one general aspect, the present invention relates to a replacement cover for muscle sheath. The replacement cover comprises at least one layer of human amnion and chorion tissues and has a shape appropriate for enclosing a torn or injured muscle.

In another general aspect, the present invention relates to a method of preparing a replacement cover for muscle sheath. The method comprises drying an allograft comprising at least one layer of human amnion and chorion tissues on a frame of a shape appropriate for enclosing a torn or injured muscle. In one embodiment of the present invention, the frame is rigid or semi rigid.

Another general aspect of the invention relates to a method of performing a surgical repair of a muscle in a subject. The method comprises:
(a) surgically repairing the muscle to obtain a surgically repaired muscle in the subject; and
(b) covering a damaged site of muscle with at least one of an amniotic fluid and a replacement cover for muscle sheath prior to wound closing,
   wherein the damaged site of muscle comprises at least one of the surgically repaired muscle and a damaged muscle sheath, the replacement cover comprises at least one layer of human amnion and chorion tissues, and the replacement cover has a shape appropriate for enclosing the muscle.

Yet another general aspect of the invention relates to a kit, comprising:
(a) a replacement cover for muscle sheath; and
(b) instructions on how to use the replacement cover in a surgical repair of a muscle,

wherein the replacement cover comprises at least one layer of human amnion and chorion tissues and has a shape appropriate for enclosing the muscle.

In a preferred embodiment of the present invention, the human amnion and chorion tissues used in the present invention are obtained by a process comprising:
(a) obtaining informed consent from pregnant females;
(b) conducting risk assessment on the consented pregnant females to select an amnion donor;
(c) procuring after birth placenta from the amnion donor; and
(d) obtaining the human amnion and chorion tissues from the placenta.

Other aspects, features and advantages of the invention will be apparent from the following disclosure, including the detailed description of the invention and its preferred embodiments and the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 illustrates replacement covers for muscle sheath of various shapes according to embodiments of the present invention.

### DETAILED DESCRIPTION OF THE INVENTION

Unless defined otherwise, all technical and scientific terms used herein have the same meaning as commonly understood to one of ordinary skill in the art to which this invention pertains. In this application, certain terms are used, which shall have the meanings as set in the specification. It must be noted that as used herein and in the appended claims, the singular forms “a,” “an,” and “the” include plural reference unless the context clearly dictates otherwise.

In one general aspect, embodiments of the present invention relate to a replacement cover for muscle sheath. The replacement cover comprises at least one layer of human amnion and chorion tissues and has a shape appropriate for enclosing a muscle, such as a torn, ruptured, injured, deformed or pathological diseased muscle, or a surgically repaired muscle.

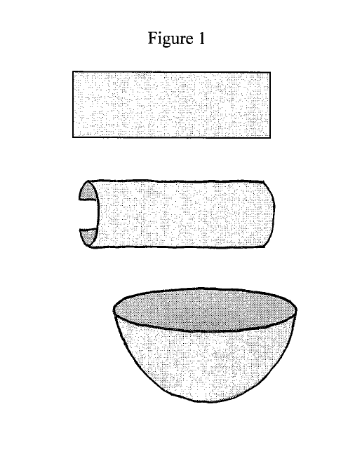

The replacement cover can be of various shapes, lengths and diameters, and thickness to fit the various muscles in the body. Exemplary shapes of the replacement cover include, but are not limited to, flat sheets, cylindrical or tubular shapes, concave bowls or curved sheets, see, e.g., FIG. 1. In one embodiment, the replacement cover is generally cylindrical with a C-shaped cross-section to allow for ease of implantation over the torn muscle.

In one embodiment of the present invention, the replacement cover further comprises a frame, which can be flexible, rigid or semi rigid. Preferably, the frame is a rigid or semi rigid frame. The thickness of the frame can be between 0.5 mm to 2 mm and the length and circumference are the same as the allograft tissue(s) bonded to it. In one embodiment, the frame is disposable. In another embodiment, the frame is implantable and resorbable. When a frame is used, in the case of either dry, wet or frozen allograft tissues, it facilitates the allograft tissues to be implanted over the torn or injured muscle.

In one embodiment of the present invention, for the repair of a larger muscle, an allograft comprising at least one layer of human amnion and chorion tissues is dried into a flat sheet, with or without a rigid or semi rigid frame, and the dried flat sheet is used as the replacement cover.

In another embodiment of the present invention, for the repair of a smaller torn muscle, an allograft comprising at least one layer of human amnion and chorion tissues is processed in a way that creates a tubular construct, preferably rigid or semi-rigid, which can cover a muscle. Then, by the process of rehydration, the allograft can adhere to the muscle.

In yet another embodiment of the present invention, for the repair of a smaller muscle, an allograft comprising at least one layer of human amnion and chorion tissues is attached to a frame, preferably a resorbable rigid or semi rigid polymer frame (in the case of either dry, wet or frozen human allograft tissues), which allows the allograft membrane to be implanted over injured muscle.

In one embodiment of the present invention, one or more corners of the replacement cover are rounded or flatted to prevent the corners from catching during implantation. In view of the present disclosure, any method known to those skilled in the art can be used to make the corners of the replacement cover round or flatten.

In one embodiment of the present invention, the replacement cover can carry one or more therapeutic agents, such as morphogenic proteins, small molecule compounds, pharmaceutical agents, anti-microbial agents, anti-inflammatory agent, agents that prevent scarring, adhesions and tethering of internal tissue of the torn muscle or the surgery site, analgesics, etc., to further improve the performance and reduce the complications of torn muscle or its surgical repair. Examples of the growth enhancing agent include, but are not limited to, growth hormone, insulin like growth factor I, keratinocyte growth factor, fibroblast growth factor, epidermal growth factor, platelet derived growth factor and transforming growth factor, and a combination of any of the foregoing.

In another general aspect, embodiments of the present invention relate to a method of preparing a replacement cover for muscle sheath. The method comprises drying an allograft comprising at least one layer of human amnion and chorion tissues on a frame of a shape appropriate for enclosing a muscle, such as a torn, ruptured, injured, deformed or pathological diseased muscle, or a surgically repaired muscle. The frame can be flexible, rigid or semi-rigid. In one embodiment of the present invention, the method comprises drying an allograft comprising at least one layer of human amnion and chorion tissues on a rigid or semi rigid frame of the shape selected from the group consisting of flat sheets, cylindrical or tubular shapes, concave bowls or curved sheets.

In an embodiment of the present invention, when a disposable frame is used, the dried tissue retains the shape of the frame when removed from the frame or could be packaged and sterilized with a disposable frame to retain its shape prior to use. The disposable frame can be removed and discarded prior to the use of the tissue. The disposable frame can be longer than the tissue for ease of handling and removal.

In another embodiment of the present invention, the allograft in the replacement cover is reinforced with an implantable and resorbable polymer frame of a shape appropriate for enclosing a torn or injured muscle. The frame can be flexible, rigid or semi rigid, preferably rigid or semi rigid. This implantable and resorbable frame could be a mesh or a solid frame with several holes throughout.

The allograft, such as human allograft comprising one or more layers of amnion and/or chorion tissues, is bonded to the frame by various methods in view of the present disclosure, such as, drying the tissue on the frame, using a resorbable adhesive, keeping the tissue wet and laying it on the frame, or freezing the tissue on the frame.

Another general aspect of the present invention relates an improved method of repairing a muscle, such as a torn, ruptured, injured, deformed or pathological diseased muscle in a subject. The method comprises:
(a) surgically repairing the muscle to obtain a surgically repaired muscle in the subject; and
(b) covering a damaged site of muscle with at least one of an amniotic fluid and a replacement cover for muscle sheath prior to wound closing,
   wherein the damaged site of muscle comprises at least one of the surgically repaired muscle and a damaged muscle sheath, the replacement cover comprises at least one layer of human amnion and chorion tissues, and the replacement cover has a shape appropriate for enclosing the muscle.

The amniotic fluid and the replacement cover for muscle sheath can be applied to the damaged site of muscle individually or in combination. The damaged site of muscle can result from an injury, such as a torn, ruptured, injured, deformed or pathological diseased muscle. The damaged site of muscle can also result from the surgery repair. Preferably, the amniotic fluid is processed so that it has a relatively high viscosity for ease of application and for remaining in the desired area after the application.

In one embodiment of the present invention, both the amniotic fluid and the replacement cover are applied to the damaged site of muscle, preferably, the amniotic fluid has a relatively high viscosity.

In another embodiment of the present invention, only the amniotic fluid is applied to the damaged site of muscle, preferably the amniotic fluid has a relatively high viscosity.

In a preferred embodiment of the present invention, a replacement cover for muscle sheath according to an embodiment of the present invention is applied over a damaged site of muscle during a surgical repair of a muscle, preferably after the torn, ruptured, injured, deformed or pathological diseased muscle is sutured or repaired.

The improved method can be applied to any procedure of surgical repair of any torn, ruptured, injured, deformed or pathological diseased muscle in view of the present disclosure. The surgery can be open surgery or percutaneous surgery. For example, during an open surgery, an incision is made in the skin over the identified injury site. The torn or injured muscle is inspected and stitched or sutured together. A replacement cover for muscle sheath according to an embodiment of the present invention is placed on or around the sutured muscle then hydrated. After hydration, the replacement cover adheres to the sutured muscle. Methods of the present invention also apply to a percutaneous surgery, where several small incisions rather than one large incision are made in the skin over the identified injury site.

The circumference of the replacement cover can be slightly greater than half a full circle to allow ease of implantation over the sutured muscle. The circumference of the replacement cover can be larger than the muscle it will be implanted on so that when hydrated it will fully encase the muscle.

The replacement cover according to an embodiment of the present invention can be used in various surgical repairs of muscles, including, but not limited to, incision of muscle, fascia and bursa; fasciotomy; excision of lesion of muscle, tendon, fascia, and bursa; suture of muscles, tendon, and fascia primary repair of muscle ruptures; reconstruction of muscle and tendon; transfer or transplantation of muscle and tendon; plastic operations on muscles, tendon and fascia; tendon pulley reconstruction; muscle and tendon lengthening; myotendinous lengthening; freeing of adhesions of muscle, tendon, fascia, and bursa; and tenolysis.

According to an embodiment of the method of the present invention, a replacement cover comprising an allograft of amniotic membrane is positioned into the place between abutting surfaces. The allograft is placed between the surfaces that may adhere. Muscle belly's outer surface (epimyceum) is covered with the allograft. The allograft is placed in a manner that it separates the tissues of the body that have been traumatized by surgery from remaining undisturbed tissues of the body (fascia).

According to another embodiment of the present invention, the replacement cover is placed in a manner that it separates two traumatized tissues of the body. The separation in this manner reduces the formation of adhesions between tissue surfaces. Once the allograft is properly positioned at the desired site, the surgeon can extend it beyond the incision or traumatized area to facilitate its coverage around the tissue contours. The allograft is then allowed to contact the desired site and held by surface tension. The allograft conforms well to moist tissues and can be used in the presence of blood.

According to another embodiment of the present invention, the allograft is sutured into place, e.g., with #6.0 nylon suture. The allograft is then hydrated with sterile saline. The surgical site is then closed according to the standard technique of the surgeon.

In another embodiment of the present invention, a construct comprising a layer of amnion is used to cover a skin incision resulting from the surgery. The allograft patch can be of any size suitable for covering the sutures or other type of tissue injuries at skin incision.

Preferably, a relatively thick layer of allograft is used to cover the skin incision. In one embodiment of the invention, the allograft patch has a thickness of about 2 mm to 4 mm. It can have multiple layers of amnion or a combination of multiple layers of amnion and chorion in any combination of amnion and chorion.

The present invention overcomes shortcomings of the prior art by making human allograft membranes usable as surgical implants to repair torn muscles or damaged muscle sheaths during surgery.

Amnion has a complete lack of surface antigens, thus does not induce an immune response when implanted into a ‘foreign’ body, which is in contrast to most other allograft implants. Amnion also markedly suppresses the expression of the pro-inflammatory cytokines, IL-1α and IL-1β (Solomon et al., 2001, Br J. Ophthalmol. 85(4):444-9) and produces natural inhibitors of matrix metalloproteases (MMPs) expressed by infiltrating polymorphonuclear cells and macrophages. Hao et al., 2000, Cornea, 19(3):348-52; Kim et al., 2000, Exp Eye Res. 70(3):329-37). Amnion also down-regulates TGF-β and its receptor expression by fibroblasts leading to the ability to modulate the healing of a wound by promoting tissue reconstruction. Furthermore, amnion and chorion contain antimicrobial compounds with broad spectrum activity against bacteria, fungi, protozoa, and viruses for reduced risk of post-operative infection. All of these characteristics of amnion make it a potential allograft candidate to be used in treating torn muscles.

Human allograft amnion and chorion have the ability to prevent scarring, reduce inflammation, inhibit microbial infection and improve healing. Repairing torn muscles, however, requires the surgeon to work in very tight spaces and repairing the muscle sheath is extremely difficult. Surgeons who would attempt to repair the muscle sheath with a replacement membrane could encounter several problems. Curving a flat sheet around a small muscle at the surgical site is extremely difficult for the surgeon.

By creating a rigid or semi-rigid, curved shape which mimics the size and characteristics of a human muscle from human allograft amnion and/or chorion membrane material which has the ability to reduce adhesions, scar formation while also reducing inflammation and risk of post-operative infection would simplify implantation.

Amnion fluid or tissues used in the present invention can be prepared from birth tissue procured from a pregnant female. Informed consent is obtained from a pregnant female by following guidelines as promulgated by the American Association of Tissue Banks and consistent with guidelines provided the Food and Drug Administration: a federal agency in the Department of Health and Human Services established to regulate the release of new medical products and, finally, if required by an established review body of the participating hospitals or institutions. The pregnant female is informed that she will be subject to risk assessment to determine if she is qualified as a birth tissue donor. She will also be informed of the tests for the risk assessment. The pregnant female is further informed that, if she is selected as a birth tissue donor based on the risk assessment, her birth tissues, such as placenta and amniotic fluid, may be collected at birth, tested and processed for medical uses.

The informed consent includes consent for risk assessment and consent for donation of birth tissues.

Risk assessment is conducted on a pregnant female with informed consent to evaluate her risk factors for communicable diseases, such as human immunodeficiency virus (HIV), hepatitis B virus (HBV), hepatitis C virus (HCV), cytomegalovirus (CMV), human T-lymphotropic virus (HTLV), syphilis, etc. Medical and social histories of the pregnant female, including physical exam record, and/or risk assessment questionnaire, are reviewed. Pregnant females with high risk factors for the communicable diseases are excluded.

Consent to draw blood at time of delivery and 1 to 12 months post delivery is obtained from pregnant females with low risk factors for the communicable diseases. Screening tests on communicable diseases, such as HIV 1 and 2, HCV, HbCore, syphilis, HTLV I/II, CMV, hepatitis B and C, are conducted by conventional serological tests on the blood sample obtained at birth. The initial screening tests are preferably completed within 7 days after birth. Preferably, the screening tests are conducted again on a second blood sample collected a few months post delivery, to verify the previous screening results and to allow for detection of communicable disease acquired shortly before birth, but are shown as “negative” on the previous screening tests. The second blood sample can be collected 1-12 months, preferably 6 months, post birth.

Only pregnant females with informed consent who are tested negative for the communicable diseases are approved as birth tissue donor. In a preferred embodiment, only pregnant females with informed consent who are tested negative for the communicable diseases in both screening tests with the blood sample drawn at birth and the blood sample drawn 6 months post delivery are approved as birth tissue donor.

Sterile techniques and procedures should be used as much as practically possible in tissue handling, e.g., during tissue procurement, banking, transfer, etc., to prevent contamination of the collected tissues by exogenous pathogens.

Only birth tissues procured from the approved birth tissue donors are subject to the collection and subsequent processing. Birth tissues, such as placenta and amniotic fluid, are recovered from the delivery room and are transferred to a location in a sterile container, such as a sterile plastic bag or bottle. Preferably, the tissues are transferred in a thermally insulated device at a temperature of 4° to 28° C., for example, in an ice bucket.

According to an embodiment of the invention, shortly after its expulsion after birth, a suitable human placenta is placed in a sterile bag, which is placed in an ice bucket, and is delivered to another location. The placenta is rinsed, e.g., with sterile saline, to removed excessive blood clots. Preferably, the placenta is subject to aseptic processing, for example, by including one or more antibiotics, such as penicillin and/or streptomycin, in the rinse. The aseptically processed placenta is stored in a controlled environment, such as hypothermic conditions, to prevent or inhibit apoptosis and contamination.

The processed placenta is placed in a sterile container, such as one made of triple sterile plastic bags, packed in wet ice, and shipped to a location for subsequent processing via overnight courier. The placenta is shipped together with release documents for processing. For example, each shipment must include technical approval to process based upon a satisfactory review of the criteria for donor selection and donor approval. The shipment must also include results on screening of communicable diseases. Preferably, the shipment includes medical director review and approval of donor eligibility/suitability.

Upon receiving the shipment and a satisfactory review of the accompanying release documents, the amnion is separated from the chorion and other remaining tissues of placenta using methods known in the art in view of the present disclosure. For example, the amnion can be stripped off mechanically from the placenta immersed in an aseptic solution, e.g., by tweezers. The isolated amnion can be stored in a cryoprotective solution comprising a cryoprotective agent, such as dimethyl sulfoxide (DMSO) and glycerol, and cryopreserved by using a rapid, flash-freeze method or by controlled rate-freeze methods. Preferably, the isolated amnion is treated with one or more antibiotics, such as penicillin and/or streptomycin, prior to cryopreservation. The chorion can also be separated from the other tissues, preserved and stored for future use.

The isolated amnion is a tough, transparent, nerve-free and nonvascular sheet of membrane. It can be dried or lyophilized using various methods. For example, it can be dried over a sterile mesh, for example, by being placed on a sterile nitrocellulose filter paper and air dried for more than 50 minutes in a sterile environment. It can also be dried or lyophilized over other form of supporting material, which would facilitate the subsequent manipulation of the amnion, such as sterilizing, sizing, cataloging, and shipping of the amnion.

The present invention encompasses a kit comprising at least one of an amniotic fluid and a replacement cover for muscle sheath and instructions on how to use the amniotic fluid and the replacement cover in a surgery to repair a muscle. The replacement cover comprises at least one layer of human amnion and chorion tissues and is adapted to a shape appropriate for enclosing the muscle. One or more corners of the replacement cover can preferably be rounded or flatted to prevent the corner from catching during implantation. In a preferred embodiment, the kit comprises a plurality of replacement covers for muscle sheaths, and at least two of the plurality of replacement covers have different shapes or sizes suitable for covering different damaged sites of muscles. The replacement cover can further comprise one or more therapeutically active agents as those described above.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/We claim:

1. A replacement cover for muscle sheath, comprising at least one layer of human amnion and chorion tissues, wherein the replacement cover has a shape appropriate for enclosing a muscle.

2. The replacement cover of claim 1 further comprising a frame that is disposable or implantable and resorbable.

3. The replacement cover of claim 2, wherein the frame is rigid or semi rigid.

4. The replacement cover of claim 1, having a shape selected from the group consisting of a flat sheet, a cylindrical shape or tubular shape, a generally cylindrical shape with a C-shaped cross-section, and a concave bowl or a curved sheet.

5. The replacement cover of claim 1, having one or more rounded or flattened corners

6. The replacement cover of claim 1, further comprising one or more therapeutic agents selected from the group consisting of morphogenic proteins, small molecule compounds, pharmaceutical agents, anti-microbial agents, anti-inflammatory agent, agents that prevent scarring, adhesions and tethering of internal tissue of the muscle or the surgery site, and analgesics.

7. The replacement cover of claim 6, wherein the therapeutic agent is selected from the group consisting of growth hormone, insulin like growth factor I, keratinocyte growth factor, fibroblast growth factor, epidermal growth factor, platelet derived growth factor, transforming growth factor, and a combination thereof.

8. The replacement cover of claim 1, wherein the human amnion and chorion tissues are obtained using a process comprising:
a. obtaining informed consent from pregnant females;
b. conducting risk assessment on the consented pregnant females to select an amnion donor;
c. procuring after birth placenta from the amnion donor; and
d. obtaining the human amnion and chorion tissues from the placenta.

9. A method of preparing a replacement cover of claim 2, the method comprising drying an allograft comprising at least one layer of human amnion and chorion tissues on the frame.

10. A method of performing a surgical repair of a muscle in a subject, comprising:
(a) surgically repairing the muscle in the subject to obtain a surgically repaired muscle in the subject; and
(b) covering a damaged site of muscle with at least one of an amniotic fluid and a replacement cover for muscle sheath prior to wound closing,
wherein the damaged site of muscle comprises at least one of the surgically repaired muscle and a damaged muscle sheath, the replacement cover comprises at least one layer of human amnion and chorion tissues, and the replacement cover has a shape appropriate for enclosing the muscle.

11. The method of claim 10, further comprising applying one or more allografts comprising at least one layer of human amnion and chorion tissues over one or more suture lines and incisions resulting from the surgical repair to form a cover and barrier over the suture lines and the incisions.

12. The method of claim 10, wherein the surgical repair comprises one or more selected from the group consisting of incision of muscle, fascia and bursa; fasciotomy; excision of lesion of muscle, tendon, fascia, and bursa; suture of muscles, tendon, and fascia primary repair of muscle ruptures; reconstruction of muscle and tendon; transfer or transplantation of muscle and tendon; plastic operations on muscles, tendon and fascia; tendon pulley reconstruction; muscle and tendon lengthening; myotendinous lengthening; freeing of adhesions of muscle, tendon, fascia, and bursa; and tenolysis.

13. The method of claim 10, wherein the replacement cover has a shape selected from the group consisting of a flat sheet, a generally cylindrical or tubular shape with a C-shaped cross-section, and a concave bowl or curved sheet.

14. The method of claim 10, wherein the replacement cover has one or more rounded or flattened corners.

15. The method of claim 10, wherein the replacement cover further comprises a frame that is disposable or implantable and resorbable.

16. The method of claim 10, wherein the replacement cover comprises at least one layer of human amnion and at least one layer of chorion.

17. The method of claim 10, further comprising administering to the subject one or more therapeutic agents selected from the group consisting of morphogenic proteins, small molecule compounds, pharmaceutical agents, anti-microbial agents, anti-inflammatory agent, agents that prevent scarring, adhesions and tethering of internal tissue of the torn muscle or the surgery site, and analgesics.

18. The method of claim 10, wherein the amniotic fluid and the human amnion and chorion tissues are obtained using a process comprising:
a. obtaining informed consent from pregnant females;
b. conducting risk assessment on the consented pregnant females to select an amnion donor;
c. procuring the amniotic fluid from the amnion donor;
d. procuring after birth placenta from the amnion donor; and
e. obtaining the human amnion and chorion tissues from the placenta.

19. A kit comprising:
(a) at least one of an amniotic fluid and a replacement cover for muscle sheath; and
(b) instructions on how to use the amniotic fluid and the replacement cover in a surgical repair of a muscle,
wherein the replacement cover comprises an allograft comprising at least one layer of amnion and chorion tissues, and the replacement cover has a shape appropriate for enclosing the muscle.

20. The kit of claim 19, comprising a plurality of replacement covers for muscle sheath, wherein at least two of the plurality of replacement covers have different shapes or sizes.
